(19) United States
(12) Patent Application Publication
(10) Pub. No.: US 2004/0039433 A1
(43) Pub. Date: Feb. 26, 2004

(54) Therapeutic bio-energy unit

(76) Inventor: Janusz Wodnicki, Krakow (PL)

Correspondence Address: Miroslaw Paczulski, Patrade Int'l, 2621 Hartman Drive, center Valley, PA, 18034, US

(21) Appl. No.: 10/363,980

(22) PCT Filed: Sep. 14, 2001
(86) PCT No.: PCT/PL01/00077

(30) Foreign Application Priority Data
Sep. 26, 2000 (PL) P-342829

(51) Int. Cl. A61N 1/04

(52) U.S. Cl. 607/115

(57) ABSTRACT

A therapeutic bio-energy unit has at least one energising module, which consists of at least two spirals (21, 22), one of which has an external coil (20), which leads to a segment (23) showing slight curvature. The segment (23) of slight curvature leads next to the closing curve (26) which is convex towards the spiral (22) and which circles two curvilinear energising elements (24, 25), which are convex towards the closing curve (26), and which are the

### BACKGROUND OF THE INVENTION

1. Field of the Invention

The present invention relates to a therapeutic bio-energy unit used in therapy through application of magnetic or electromagnetic field.

2. Brief Description of the Background of the Invention Including Prior Art

Polish patent description No. 124 434 shows a therapeutic unit relying on pulsating electric currents. The unit consists of the relaxation generator linked—through formation set—with the amplitude modifier powered with alternate current, and two terminal current amplifiers, which are connected to a pair of procedure electrodes applied directly on the patient's body. The terminal current amplifiers of the unit are galvanically separated from each other, and from the power system supplying the amplitude modifier.

Polish patent description No. 154 986 shows the method and equipment unit applied in non-intervention diagnosis of human internal organs, through the measurement of electric properties displayed by particular sections of the auricle, which is conceptually close to RYODARAK method. Following the diagnosis of particular human body organs—their activity is stimulated by electric impulses.

Polish invention design description P-302107 presents also therapeutic woollen bedclothes with inserted magnetic bio-stimulators, i.e. the magnetic-dielectric tape with ferrite warp. The selection of applied materials and construction of the cloth provides the user with stable magnetic field of 0.2-0.3 Oe, stable temperature and body ventilation, as well as negative electrostatic potential. The cover blanket and the sheet are made of two spreads of “velour”-type cloth, set together with their warps and lined with silk finish. Both the cover blanket and the sheet are covered with symmetrically attached stripes of magnetic-dielectric tape. The pillow is supplied with one stripe of magnetic-dielectric material. In effect the magnetic field envelops the entire body of sleeping person.

All the above presented equipment units have the common feature of generating a magnetic or electromagnetic field. Alternatively, through difference of potentials between particular sections of human body, the migration of additional electric load is caused.

Polish patent description No. 166628 shows also an internal shoe sole, reflecting the basic flat external shoe sole geometry, made of elastic foam material. In its upper section the internal sole has numerous lifted pads in the area of reaction spheres of natural foot-sole.

Polish invention design description P-297547 indicates also a sole of therapeutic shoe, consisting of two elastic layers, separated by rubber spikes interspersed on the entire area of the sole.

The above defined inserts and soles exercise mechanical impact upon the reaction spheres of the foot, and may result in skin irritations.

Polish invention design description P-302807 shows a module-structured geometrical de-radiation unit. P-302809 shows a bio-energy generator, and P-302808 a bio-energy bracelet. Their common feature is the fact that they contain less than one layer of radiation transferring material, where the phenomenon of total internal light reflection is used.

### SUMMARY OF THE INVENTION

1. Purposes of the Invention

It is an object of the present invention to provide a unit which is not harmful and which can be used more easily.

This and other objects and advantages of the present invention will become evident from the description which follows.

2. Brief Description of the Invention

The therapeutic bio-energy unit according to the present invention has at least one energising module, which is situated in direct proximity of human organism, or is attached to it. The unit consists of at least two spirals. At least one of them has an external coil, which leads to a segment with slight curvature. This segment may next proceed into a closing curve, the convexity of which is directed towards the spiral, and which circles two curvilinear energising elements, convex towards the closing curve, and which are included as elements of energising module. The energising module is advantageously paired with its mirror reflection, on the module which is a homothetic transformation with the centre in 0 point and the scale different from zero.

It is recommendable for the energising module to be made of diamagnetic material, and to be placed in stabilising layer, which can be adjacent on one side to the protective layer, and on the other side—the cover layer.

The energising module can be built into the household appliances or their parts, which are in close vicinity to human body. It can also be placed in clothes or their elements, additional arm or body bands, shoes or shoe inserts, the supports of seats and armchairs, in carpeting and other elements of public transport vehicles, in the elements of game machines, in sections of electric appliances, telecommunication and computer equipment, including the frame, keyboard, mouse, mouse pad, as well as laptops and mobile phones.

It is recommended that the setting layer, the protective layer, or the cover layer be supplied with magnetic or electromagnetic correctors, generating a magnetic or electromagnetic field.

Similar correctors can be placed (together with the energising module) in clothes or their elements, or additional body bands, in shoes, shoe inserts, the supports of seats and armchairs, in carpeting and other elements of public transport vehicles, in the elements of game machines, in sections of electric appliances, telecommunication and computer equipment, including the frame, keyboard, mouse, mouse pad, as well as laptops and mobile phones.

It is recommended that the energising module be placed in a sticker, which has one surface with adhesive properties, and which can be attached (together with energising module) to game machines, electric appliances, telecommunication and computer equipment, including the frames, keyboards, computer mice, laptops, mouse pads, and mobile phones.

In the case of using only the energising modules, the described bio-energy unit has the advantage that it does not generate any additional electromagnetic field. Instead, it reinforces the natural electromagnetic field of the Earth, and the field from outer space, as well as the electromagnetic field of human being, influencing the living cells.

The novel features which are considered as characteristic for the invention are set forth in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

The present invention is illustrated in preferable embodiments in the accompanying drawings in which

FIG. 1 shows a double-spiral energising module,

FIG. 2—a four-element energising module,

FIG. 3—the set of two double-spiral energising modules in mirror positions,

FIG. 4—another double-spiral energising module,

FIG. 5—another set of double-spiral energising modules,

FIG. 6—bio-energy carpet pad,

FIG. 7—bio-energy carpet pad together with anti-static cover,

FIG. 8—side view of the bio-energy unit,

FIG. 9—top view of the bio-energy unit,

FIG. 10—another bio-energy unit,

FIG. 11—furniture units with in-built energising modules,

FIG. 12—mattress with in-built energising modules,

FIG. 13—an overall with in-built energising modules,

FIG. 14—a shoe with in-built energising module,

FIG. 15—an insert containing energising module,

FIG. 16—a steering wheel with cover including energising module and correctors,

FIG. 17—public transport vehicle supplied with energising modules and correctors,

FIG. 18—an aeroplane supplied with bio-energy modules and correctors,

FIGS. 19 and 20—computer mouse and mouse pad with energising module and correctors,

FIGS. 21 and 22—laptop and keyboard with energising module and correctors.

### DESCRIPTION OF INVENTION AND PREFERRED EMBODIMENT

The invention shall be described in detail in relation to the bio-energy carpeting pad, and in particular in relation to the backbone system, though it may also have the form of shoulder or loin system, as shown diagrammatically in the enclosed figures. In the final section, the invention shall be described in relation to subsystems and elements in public transport vehicles, the elements of game machines, sections of electric appliances, telecommunication and computer equipment, including the frame, keyboard, mouse, mouse pad, as well as laptops and mobile phones.

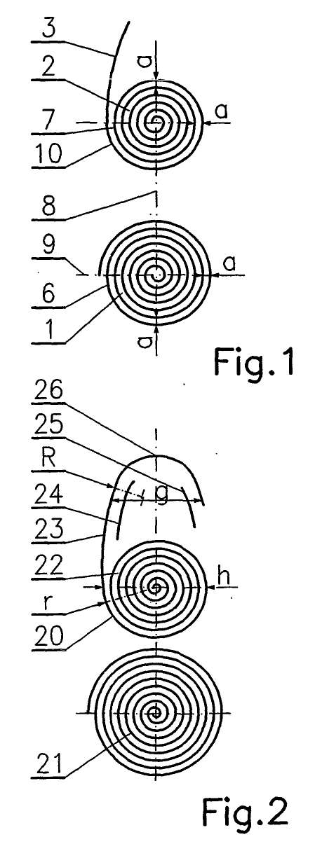

FIG. 1 shows the double-spiral energising module. Its spiral 1 has counter-clockwise (laevorotary) coil, and spiral 2 has clockwise (dextrorotary) coil. The distances a between the coils 6 of spiral 1, measured along the axes 8, 9 and the coils 7 of spiral 2 are equal. The external coil 10 of the clockwise spiral 2 leads to segment 3 having small curvature.

FIG. 2 shows a four-element energising module consisting of a counter-clockwise spiral 21 having nine coil loops, and clockwise spiral 22 having seven coil loops. Its external coil 20 leads initially to insignificantly curved interval 23, and next to the closing curve 26, which is externally convex. The closing curve 26 circles two curvilinear energising elements 24, 25, which are convex towards the closing curve 26. The radius R of interval 23 curvature is five to twenty five times larger than radius r of the external coil 20 curvature.

The curvature radii of the curvilinear energising elements 24, 25 are approximately equal to the curvature of segment 23. The closing curve 26 is terminated approx. at the level corresponding to the middle of energising element 25. The distance g between the end of energising element 25 and the closing curve 23 is approx. equal to the diameter h of the external coil 20.

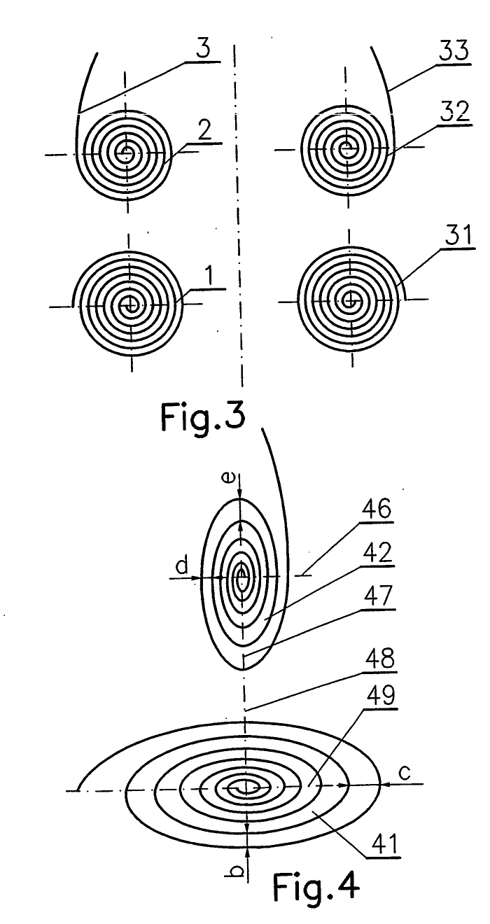

FIG. 3 shows a set of two double-spiral energising modules, where the pattern of spirals 1 and 2 is identical to the set of spirals shown in FIG. 1, while spirals 31 and 32 with the segment 33 are a mirror reflection of above set.

FIG. 4 shows a double-spiral energising module which differs from the set presented in FIG. 1 by the feature that the distance b measured along the axis 48, and the distance c measured along the coils of spiral 41 are not equal. They are also different from distances d and e between the coils of spiral 42 measured along the axes 46 and 47.

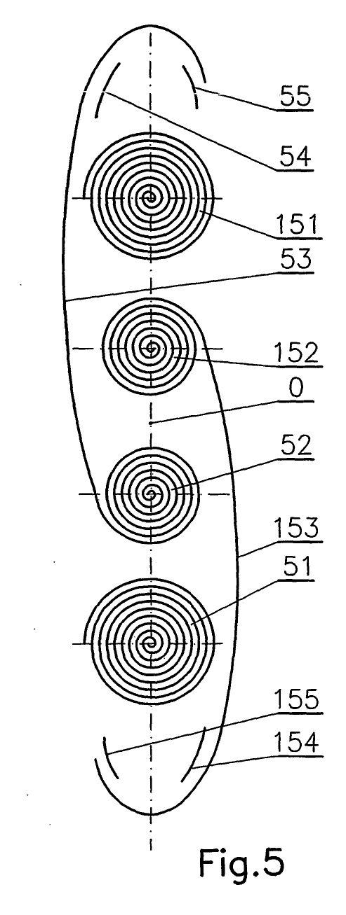

FIG. 5 shows another set of two double-spiral energising modules, where the double-spiral energising module consisting of spirals 151, 152 with the segment 153 and energising elements 154, 155 is homothetic (homothety centre 0, and scale less than zero) to the double-spiral energising module consisting of spirals 51, 52 together with the segment 53 and energising elements 54, 55.

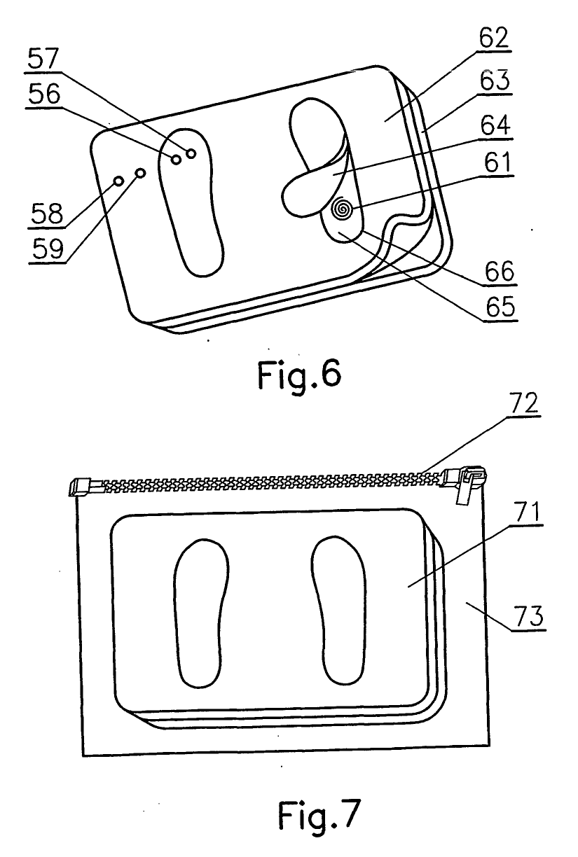

FIG. 6 shows a bio-energy carpet pad with raised upper layer 62 in one of the corners. The upper layer 62 is attached to the protective cover 63 and has incised sections 66. In every incision, the stabilising layer 65 is placed, containing spirals and other elements of energising modules 61. The stabilising layer 65 prevents the shifts of the elements of energising modules 61 when the bio-energy carpet pad is used. Upon the stabilising layer 65, the covering layer 64 is placed, in shape corresponding to the foot and incisions 66. The material for surface layer 62, the protective layer 63 and the stabilising layer 65 is produced on the basis of butadiene-styrene rubber. The cover layer 64 is produced on the basis of co-polymer of ethylene-vinyl acetate. The energising module 61 can be provided both by double-spiral energising module and four-element energising module, their combinations or pairs. The energising module is made of copper wire of any cross-section dimensions. Silver or aluminium can be also used in the energising module. The bio-energy carpet pad has the shape of rectangle with rounded corners. In other solutions the carpet pad may have any shapes; though the recommended shapes (besides the rectangle) are the triangle, circle and square. In the stabilising layer 65, in the protective layer 63 or in the cover layer 64, magnetic correctors 56 are placed, in the form of permanent magnets, or electromagnetic correctors 57, in the form of systems consisting of electromagnet, power supply, and microchip. The direction and intensity of current flowing through the coil is regulated through the microprocessor. In effect, a definite distribution of electromagnetic field is obtained within the elements in direct contact with human body. The intensity of electromagnetic field and its direction can be adjusted to individual needs. Similar magnetic correctors 58 and electromagnetic correctors 59 are placed in the protective layer 63 and the surface layer 62 in sections which are not in direct contact with human body. The magnetic correctors 58 and electromagnetic correctors 59 provide a shielding magnetic field with definite distribution of intensity and direction.

FIG. 7 shows a bio-energy carpet pad 71 placed in anti-static cover 73, closed with the fastener 72, which protects the bio-energy carpet pad 71 against accumulation of static electricity. The bio-energy carpet pad 71 encapsulated in anti-static cover 73 is protected against dirt and dust.

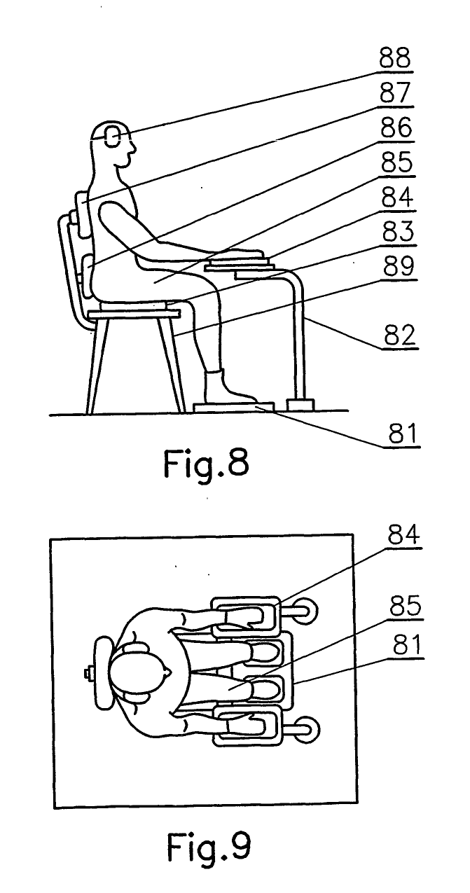

FIGS. 8 and 9 show a therapeutic bio-energy unit consisting of a bio-energy carpet pad 81, the bio-energy pad system 83 attached or placed on the chair 89, the loin and sacrum (lumbosacral). bio-energy system 86, the backbone and shoulders bio-energy system 87, the temples bio-energy system 88, and table bio-energy system attached or placed on the table 82. The shapes of bio-energy elements correspond to the body section shapes of the person 85 undergoing therapy. Bio-energy systems contain energising modules, as well as magnetic and electromagnetic correctors, as described above.

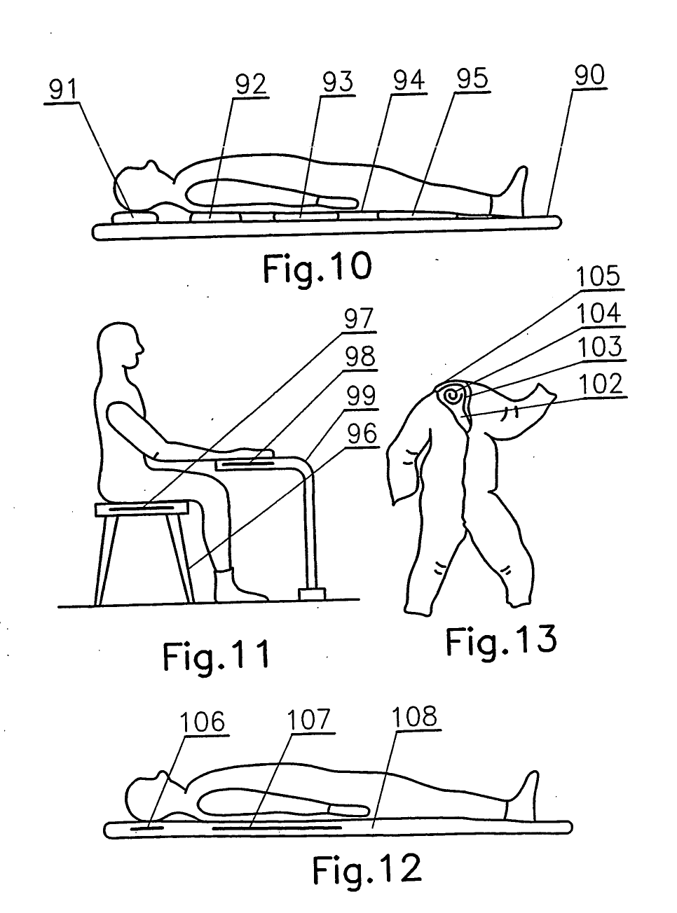

FIG. 10 shows bio-energy systems 91, 92, 93, 94, 95 attached or placed on the mattress 90. Each one of the bio-energy systems contains energising modules, magnetic and electromagnetic correctors.

FIGS. 11 and 12 show energising modules 97, 98 together with correctors in-built into the chair 96 and the stool 99, as well as energising modules 106, 107 with correctors which are placed in the mattress 108. The furniture units shown in FIGS. 8, 9 and 11 are elements supporting the computer equipment, including the computer mice and keyboards.

FIG. 13 shows an overall with backbone bio-energy system 104 and correctors. Between the surface layer 105 and the internal layer 102, the overall has a layer made of diamagnetic film 103.

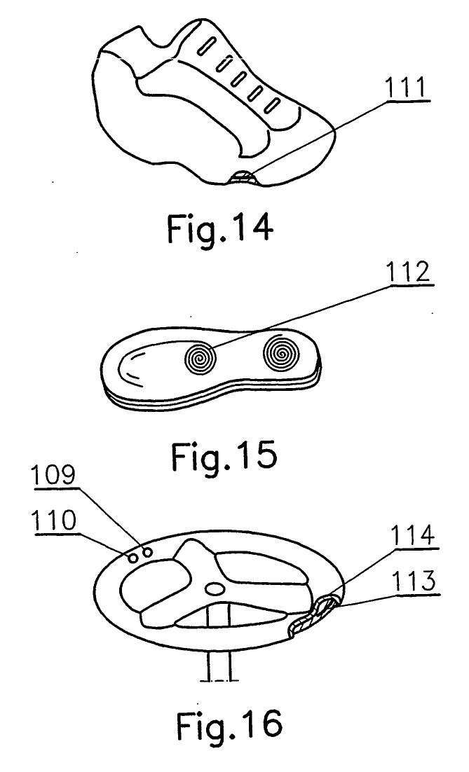

FIG. 14 shows a shoe with in-built energising module 111, magnetic or electromagnetic correctors, which are not indicated in this figure. FIG. 15 presents an insert with energising module 112 before the attachment of cover layer, which contains magnetic and electromagnetic correctors.

FIG. 16 shows a steering wheel, used in motor vehicles or game machines, with energising modules, magnetic and electromagnetic correctors, which has in-built energising modules 113, magnetic correctors 109 and electromagnetic correctors 110. Similarly, the steering wheel cover 114 contains energising modules, magnetic and electromagnetic correctors.

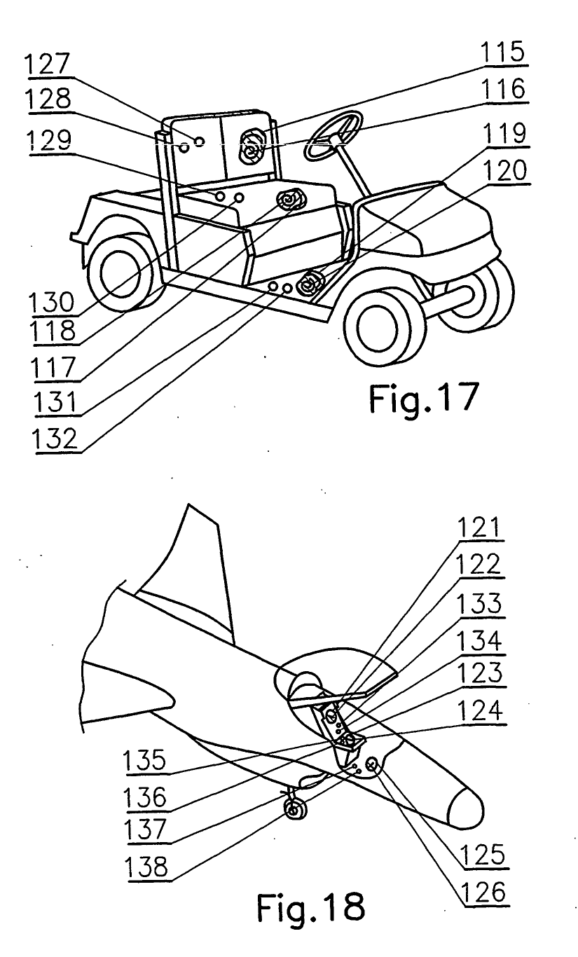

FIGS. 17 and 18 show seats and armchairs with cover elements 115, 117 and carpet pad 119, used in transport vehicles including motorcars, aeroplanes, trains, buses and ships. For example, the back support, the seat and the carpet pad have been supplied with in-built energising modules 116, 118, 120, 121, 123, 125, magnetic correctors 127, 129, 131, electromagnetic correctors 128, 130, 132, and magnetic-and-electromagnetic correctors 122, 124, 126. Similar modules and correctors have been placed in the support cover elements and seats. The energising elements and correctors have been in-built both into the elements remaining in direct contact with human body, and those not in direct contact with human body.

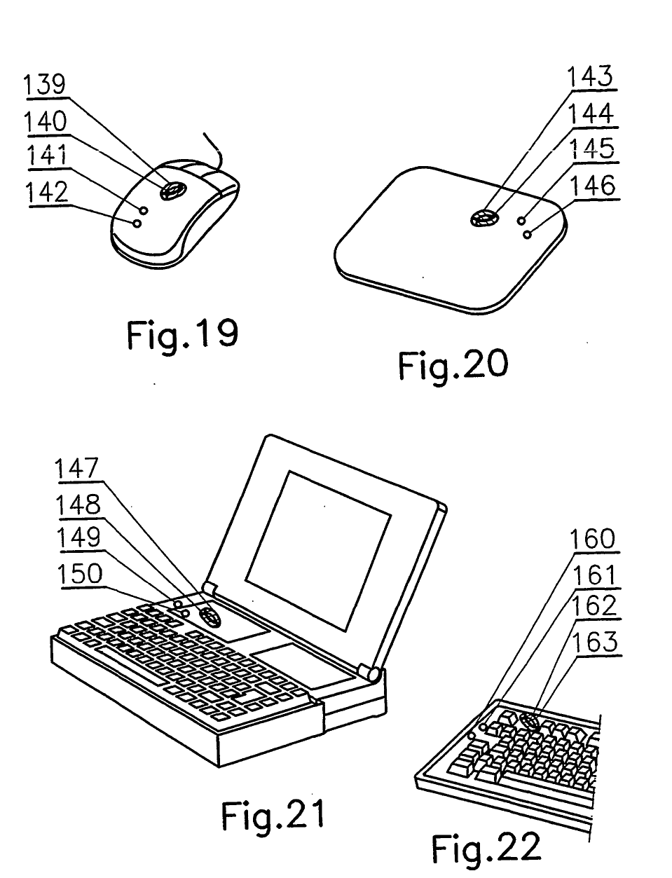

FIGS. 19, 20, 21 and 22 show the elements of computer equipment supplied with energising modules 140, 144, 148, 162, magnetic correctors 141, 145, 149, 161, and electromagnetic correctors 142, 146, 150, 160. The energising modules 140, 144, 148 are sunk into material of stickers 139, 143, 147, which are attached to the frame of computer unit. The sticker provides also the stabilising layer, preventing the deformation of energising module. In another presented solution, the energising module 162 is placed within the frame under the cover layer 163.

The therapeutic bio-energy unit has been applied successfully in the treatment of ailments of upper and lower limbs, backbone, and blood circulation system. On the basis of results obtained in research described below, it is recommended to place the energising modules in the equipment units the operation of which can lead to the contraction of veins, as it is the case with persons sitting in transportation vehicles, or using electric, telecommunication and computer devices in sitting position.

The application of therapy relying on the bio-energy unit is simple. For example the use of bio-energy carpet pad consists in spreading the carpet pad evenly and placing feet on the sections corresponding to the foot-contours.

The therapeutic bio-energy unit has been tested in BIONAT Clinic in Kraków, Poland, on the sample of 120 persons. Patients undergoing therapy showed reduced rheumatic and neurological disorders, improvement in peripheral blood circulation, leading to better blood supply in lower and upper extremities, as well as reduced psychosomatic ailments. Also, menstruation pain was reduced, and menstruation cycle was more regular.

Below, numerical results of the test are presented. The group of 31 patients reported significant reduction or elimination of rheumatic pain in lower extremities (feet, ankle joints, knees, hips, muscles). The group of 14 persons reported a significant reduction or elimination of intercostal neural pain, as well neural pain of shoulders and upper extremities. Twenty-one patients showed reduced backbone ailments (mainly degenerative). Seven patients showed improved blood supply to upper extremities. Following only one session lasting 30 minutes, five persons showed reduction of arterial blood pressure from 180/120 to 160/90, and reduction of pulse rhythm from 110/min to 95/min. Following one 20 minutes session, a significant improvement was shown in the group of 52 patients with psychosomatic symptoms, having the character of general “low feeling”, nervousness, increased muscular tension and emotional tenseness.

Older patients showed activation of life forces and increased tolerance to effort.

It will be understood that each of the elements described above, or two or more together, may also find a useful application in other types of therapeutic bio-energy units differing from the types described above.

While the invention has been illustrated and described as embodied in the context of a therapeutic bio-energy unit with spirals, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A therapeutic bio-energy unit comprising at least one energising unit situated in direct vicinity of human organism, or being direct contact with human body, and having at least two spirals, characterized in that, at least one spiral (1, 2, 21, 22, 31, 32, 41, 42, 51, 52, 151, 152) has an external coil (10, 20) with a slightly curved segment (3, 23) ended with a closing curve (26), which is convex outwards the spiral (22), and which circles two curvilinear energising elements (24, 25) being convex towards the closing curve (26).

2. The therapeutic bio-energy unit according to claim 1, characterized in that, said spirals (1, 2, 21, 22, 31, 32, 41, 42, 51, 52, 151, 152), two curvilinear energising elements (24, 25) are electrically separated.

3. (amended) The therapeutic bio-energy unit according to claim 1, characterized in that, at least one energising module is paired with a mirror energising module being a mirror reflection of said energising module.

4. (amended) The therapeutic bio-energy unit according to claim 1, characterized in that, at least one energising module is paired with a transformed energising module being a homothetic transformation of said energising module with the centre 0 and a scale different from zero.

5. The therapeutic bio-energy unit according to claim 1, characterized in that, said energising module is made of diamagnetic material and is placed in a stabilising layer (65).

6. The therapeutic bio-energy unit according to claim 1, characterized in that, said energising module is made of diamagnetic material and is placed in a stabilising layer (65) adjacent to a protective layer (63) on one side and on the other side to a surface layer (62).

7. The therapeutic bio-energy unit according to claim 1, characterized in that, said energising module (97, 98, 106, 107) is built into equipment units (82, 89, 90, 96, 99, 108) used as household appliances, or their elements remaining in close vicinity to humans.

8. The therapeutic bio-energy unit according to claim 4, characterized in that, the energising modules (104) are placed in clothes or their elements, or in additional bands worn on the body.

9. (amended) The therapeutic bio-energy unit according to claim 1, characterized in that, said energising module (111) is placed in the shoe or in the shoe insert.
Please delete claim 10

11. The therapeutic bio-energy unit according to claim 6, characterized in that, in the stabilising layer (65) and/or protective layer (63) and/or surface layer (62) magnetic correctors (56, 58), and/or electromagnetic correctors (57, 59) are placed, generating magnetic field and/or electromagnetic fields.

12. The therapeutic bio-energy unit according to claim 8, characterized in that, together with said energising module, magnetic correctors and/or electromagnetic correctors are placed in clothes or their elements, or additional bands.

13. The therapeutic bio-energy unit according to claim 9, characterized in that, together with energising module, magnetic and/or electromagnetic correctors are placed inside the shoe.

14. (amended) The therapeutic bio-energy unit according to claim 9, characterized in that, magnetic and/or electromagnetic correctors are placed inside the shoe insert together with the energising module.

15. The therapeutic bio-energy unit according to claim 4, characterized in that, the energising modules (113, 116, 118, 120, 121, 123, 125) are placed in seat supports, seats, carpet pads and other elements used in transport vehicles, and being in close vicinity to human body or in direct contact with it.

16. The therapeutic bio-energy unit according to claim 15, characterized in that, magnetic correctors (127, 129, 131) and/or electromagnetic correctors (128, 130, 132) are placed together with energising modules (113, 116, 118, 120, 121, 123, 125) in the seat supports, seats, carpet pads, and other elements used in transport vehicles and being in close vicinity to human body.

17. (amended) The therapeutic bio-energy unit according to claim 1, characterized in that, said energising module (140) is placed in a sticker (139) with an adhesive surface.

18. The therapeutic bio-energy unit according to claim 17, characterized in that, the sticker (139) together with energising module (140) is attached to game machines, electrical devices, telecommunication and computers unit, including the frames, keyboards, computer mice, laptops, mouse pads, mobile phones.

19. (amended) The therapeutic bio-energy unit according to claim 1, characterized in that, at least one energising module (144, 148, 162) is placed in elements of game machines, electrical devices, telecommunication and computers unit, including the frames, keyboards, computer mice, laptops, mouse pads, mobile phones.

20. The therapeutic bio-energy unit according to claim 19, characterized in that, magnetic correctors (141, 145, 149, 161) and/or electromagnetic correctors (142, 146, 150, 160 ) are placed together with energising module (144, 148, 162) in elements of game machines, electrical devices, telecommunication and computers unit, including the frames, keyboards, computer mice, laptops, mouse pads, mobile phones.

21. (new) The therapeutic bio-energy unit according to claim 1, characterized in that, said energising module is made of diamagnetic material and is placed in a stabilising layer (65) adjacent to a protective layer (63) on one side and on the other side to an adhesive layer (62) adhesively to human skin.
